(19) United States
(12) Patent Application Publication
(10) Pub. No.: US 2009/0030466 A1
(43) Pub. Date: Jan. 29, 2009

(54) Anterior vertebral plate with suture lock

(76) Inventor: Kevin R. Strauss, Columbia, MD (US)

Correspondence Address: PERRY E. VAN OVER & ASSOCIATES, PLLC, P.O. BOX 399, FAIRFAX, VA, 22038, US

(21) Appl. No.: 11/700,235

(22) Filed: Jan. 31, 2007

(51) Int. Cl. A61B 17/70; A61B 17/04

(52) U.S. Cl. 606/280; 606/232; 606/301; 606/304

(57) ABSTRACT

Provided is a novel system that includes an anterior vertebral body plate, suture lock bone screws, and an elongated suture lock to provide an interconnection for said bone screws, such that said system can be used for the fixation and stabilization of the cervical spine, the suture lock bone screws being connected one to the other by the elongated suture lock are prevented from unintentionally backing out of said plate. Also provided is a method of stabilizing cervical vertebrae using the disclosed system.

### BACKGROUND OF THE INVENTION

1. Technical Field

The present invention relates to devices and methods for use in orthopedic spine surgery. In particular, the present invention relates to a system that provides a low profile anterior vertebral body plate and suture lock bone screws secured and locked in place relative to the plate by at least one suture lock element, the system being used for fixation and stabilization of the spine, the suture lock screws in combination with the anterior vertebral plate and the locking suture providing a novel screw locking mechanism that requires no additional locking devices to be attached to the system.

2. Background Art

Disease, the effects of aging, or physical trauma resulting in damage to the spine has been treated in many instances by fixation or stabilization of the effected vertebra. The use of plates and screws for fixation and stabilization of the cervical vertebra has been widely accepted as a reliable practice and has proven to be highly successful clinically.

The various plates, which are attached to the anterior vertebral bodies of the spinal column by bone screws have some common features such as relatively planar body profiles that define multiple holes or slots through which the screws fit and are threaded into the bone. Various means have been used to prevent the screws from becoming loose or detached from their necessary secured or locked attachment to the vertebral plate. Among the differences between the conventionally used plates and screws is the manner in which the screws are locked into place in the hole or slot of the plate after the screws have been secured to the bone.

These conventional devices can be generally grouped into three basic categories with regard to how the screws are captured or secured in the plates.

Early plate designs were standard bone plates having holes through which screws were passed and screwed into the bone. These plates had no special provision for attaching the screws to the plate and as such were susceptible to having the screws back out of the plate over time. There have been clinically reported instances of screws backing out of these type plates with resulting surgical complications. Due to the potential and actual unreliable performance of such plates, the need for secure fixation of the screw to the plate as well as to the bone is now considered a basic requirement for vertebral plates. Due to the lack of predictable security of the screw to the plate, plates which do not secure the screw relative to the plate have fallen out of favor and virtually disappeared from use.

Efforts have been made to secure the screws relative to the plates. In one design the screw head contains a threaded hole configured to receive a set screw. After the screw has been driven into bone and the head is seated in the plate hole, the set screw is inserted into the receiving hole of the screw head. The set screw is tapered to cause the screw head to expand and frictionally engage the wall of the plate hole, thereby resisting forces which tend to cause the screw to back out. While such mechanisms have worked to some degree, the addition of a small additional part, the set screw, at the time of surgery presents the potential hazard of dropping the set screw into the surgical field or otherwise misapplying the set screw to the screw head, for example, cross threading.

An alternative approach has been to provide features in the plate, which are specifically designed to hold the screw in position once the screw is inserted through the plate and screwed into the bone. One direction taken in this effort has been to design plates that incorporate or attach individual retaining rings or snap features associated with each plate hole configured to hold the inserted screw in place relative to the plate. These plates are very common and widely used; however, an inherent problem associated with such plates is the use of the additional very small retaining elements that can become disengaged from the plate and migrate into the surrounding soft tissues. Further, difficulty experienced in accessing and disengaging the small locking elements and removing the screws from this type of plate has caused some concern for the continued use of such plates. A similar approach involves individual cams associated with each plate hole, which when rotated apply friction pressure to the screw head in an attempt to resist back out.

Another approach is to add a cover to the plate after the screws have been placed. Such a design undesirably adds steps to the surgical procedure, thickness or height to the overall construct, and is susceptible to misapplication. Yet another direction taken in this effort to provide plates with locking elements is to provide dedicated overlying features, which are attached to the top side of the vertebral plate for the purpose of covering at least a portion of the screw head and thereby holding the screw in a seated and locked position. Generally these plates are designed to provide a variety of screw covering features that are pre-attached to the plate, and which can be selectively slid or rotated into position once it has been inserted. In some devices, such covering plates cover multiple screw heads. These plates typically require an increase in the overall composite thickness of the plate in order to accommodate the additional locking feature attached to the top side of the plate. This is a particularly unacceptable condition due to the use of such plates in an area of the spine where a thin, smooth surfaced profile for the plate assembly is preferred. Another major problem with such plates is that the overlying locking element cannot always be properly positioned over the screw head if the screw shaft was, due to anatomical necessity, positioned through the plate and into the bone at an angle such that the screw head does not fully seat in the plate recess provided on the top side of the plate. Further, when one of the overlying locking elements of such a plate loosens or becomes disengaged it is then free to float away from the top side of the plate and migrate into the soft tissue adjacent to the top side of the vertebral plate.

Yet another approach is to provide machine threads in the plate hole with corresponding threads on the screw head. Thus the screw has a first, bone engaging thread on its shaft and a second machine thread on the screw head. As the thread shaft is screwed into bone the screw head approaches the plate hole and the machine thread engages the thread in the hole. Aside from the fact that there is nothing to prevent the same forces that urge the screw to back out of bone to have the same effect on the machine thread engagement, such an arrangement does not provide adequate clinical flexibility. First there is no assurance that the lead in thread of the machine thread will match up with the plate hole thread when the screw head reaches the hole, raising the possibility of cross threading. Second, the machine thread in the plate hole does not allow various angular positions between the screw and the plate, as the threads must match up and engage when the screw head reaches the hole. As to the latter point, one plate provides a threaded ring in the plate hole, which is intended to allow the head to assume a variety of angular positions.

The use of multiple plates or blocks, which are attached to adjacent vertebrae and tethered one to the other has been attempted to provide a fusionless method of treating abnormal alignment of the spine. (See U.S. Pat. No. 6,616,669, issued to Ogilvie et al.) Each of the multiple blocks is provided with multiple bores through which bone screws can be inserted to fasten each of the blocks to a respective underlying vertebral body. After fully inserting the bone screws, at least one strand or tether is threaded through channels defined in the each of adjacent multiple blocks and secured with a selected amount of tension so as to limit movement or growth of the vertebrae on the side to which the blocks are attached. While the tethers are threadably engaged with the plate, they are not attached to the bone screws and have no way of limiting the reverse torque and loosening of the screw from the bone and the plate.

There is therefore, an unfulfilled need for an anterior cervical plate system that can maintain a relatively low profile while providing the security of a locking plate system. Further there is a need for a vertebral plate that does not have additional separate small locking elements as found in conventional devices with the predictable problems of those locking elements becoming disengaged from the plate and migrating away from the top side of the plate into the surrounding soft tissue.

### SUMMARY OF THE DISCLOSURE

The present invention meets the above identified need by providing a low profile anterior vertebral body plate system, which is secured to the underlying bone using novel suture lock bone screws.

Also provided is a low profile anterior vertebral body plate system, which is secured to the underlying bone using novel suture lock bone screws having at least one suture connecting element sized and configured to receive a threadably engaged suture lock, the suture lock interacting with the suture lock bone screw so as to secure and lock the screw into a set position relative to the plate.

Also provided is a low profile anterior vertebral body plate system, which is secured to the underlying bone using multiple interconnected novel suture lock bone screws having at least one suture connecting element sized and configured to receive a threadably engaged suture lock, the suture lock interacting with the interconnected suture lock bone screws so as to secure and lock the screw into a set position relative to the plate.

Also provided is a novel low profile anterior vertebral body plate system including a plate, at least one novel suture lock bone screw, and at least one suture lock, the interaction between the plate, the suture lock bone screw and the suture lock providing a locking mechanism not requiring any additional locking elements.

Also provided is a novel low profile anterior vertebral body plate system including a plate having at least one suture anchor point, at least one novel suture lock bone screw, and at least one suture lock, the interaction between the plate, the suture lock bone screw and the suture lock providing a locking mechanism not requiring any additional locking elements.

Also provided is a novel suture lock bone screw capable of being locked into a secure position relative to an anterior vertebral body plate, the screw having a screw head with at least one suture connecting element sized and configured to receive a threadably engaged suture lock.

Also provided is a method of stabilizing spinal vertebrae, the method including providing a low profile anterior vertebral body plate, which is securely attached to the underlying bone of adjacent vertebrae using novel suture lock bone screws so as to hold one attached vertebra in a fixed position relative to the adjacent attached vertebra.

Also provided is a kit, which includes at least one low profile anterior vertebral body plate, a corresponding set of novel suture lock bone screws, and at least one suture lock, the kit not requiring any additional locking elements.

### BRIEF DESCRIPTION OF THE DRAWINGS

The foregoing and other features of the novel low profile anterior vertebral plate system will become apparent to one skilled in the art to which the disclosed system and devices relate upon consideration of the following description of exemplary embodiments with reference to the accompanying drawings, wherein:

FIGS. 1A-C respectively show top, isometric, and end views of the low profile anterior vertebral plate with four suture lock bone screws fully seated in the respective screw holes and serially interconnected one to the other by a suture lock.

FIGS. 1D-F show the same top, isometric, and end views provided in FIGS. 1A-C with the additional feature of having the suture lock threaded through suture anchors defined in the upper surface of the plate.

FIGS. 2A-C respectively show top, isometric, and cross-sectional end views of the low profile anterior vertebral plate.

FIGS. 3A-C respectively show top, isometric, and side views of the suture lock bone screw.

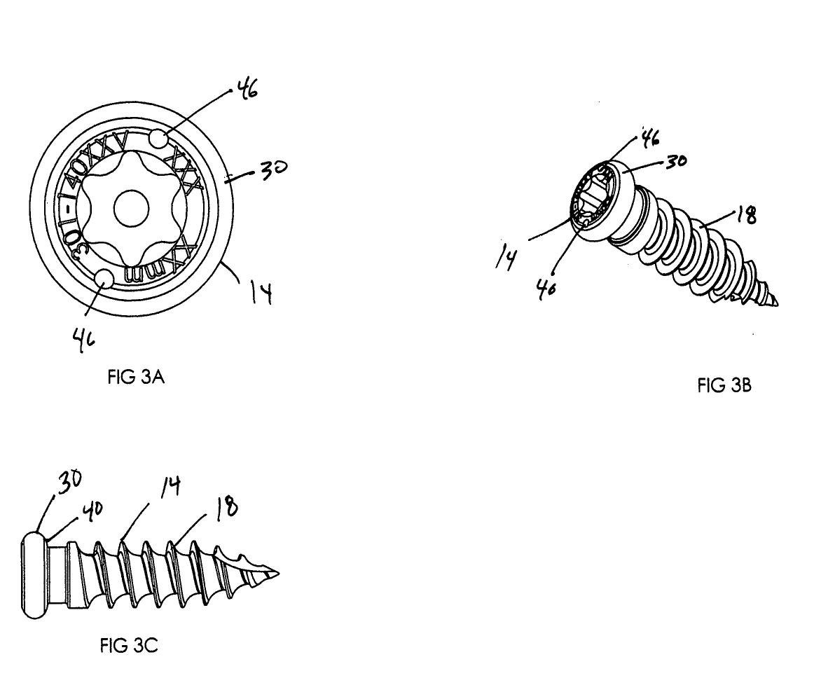

FIGS. 4A-B respectively show side and isometric views of a loose coil strand of the suture lock component of system.

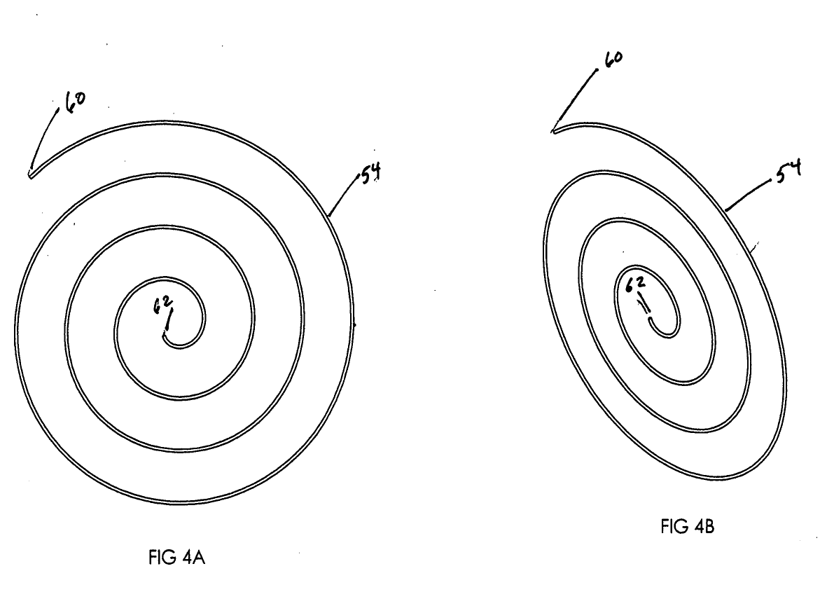

FIGS. 5A-D respectively show top, isometric, side, and alternate side views of the suture lock screw.

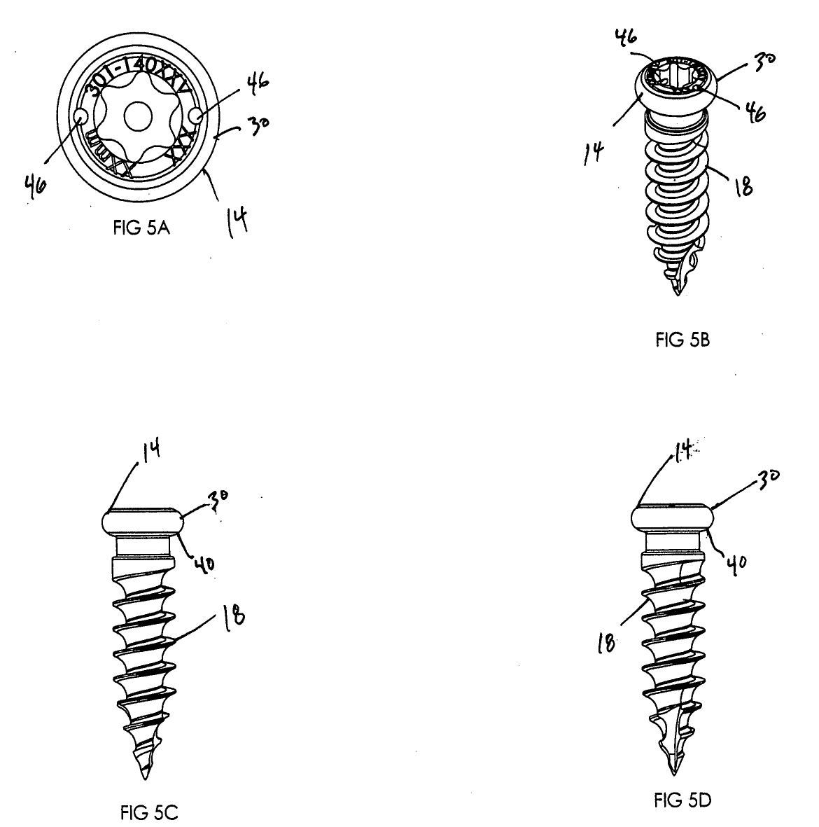

FIGS. 6A-D respectively show isometric, top, side, and cross-sectional end views of an alternative embodiment of the plate component of the system.

### DETAILED DESCRIPTION OF PREFERRED EMBODIMENTS

Detailed embodiments of the present invention are disclosed herein; however, it is understood that the following description and each of the accompanying figures are provided as being exemplary of the invention, which may be embodied in various forms without departing from the scope of the claimed invention. Thus, the specific structural and functional details provided in the following description are non-limiting, but serve merely as a basis for the invention as defined by the claims provided herewith. The device described below can be modified as needed to conform to further development and improvement of materials without departing from the inventor's concept of the invention as claimed.

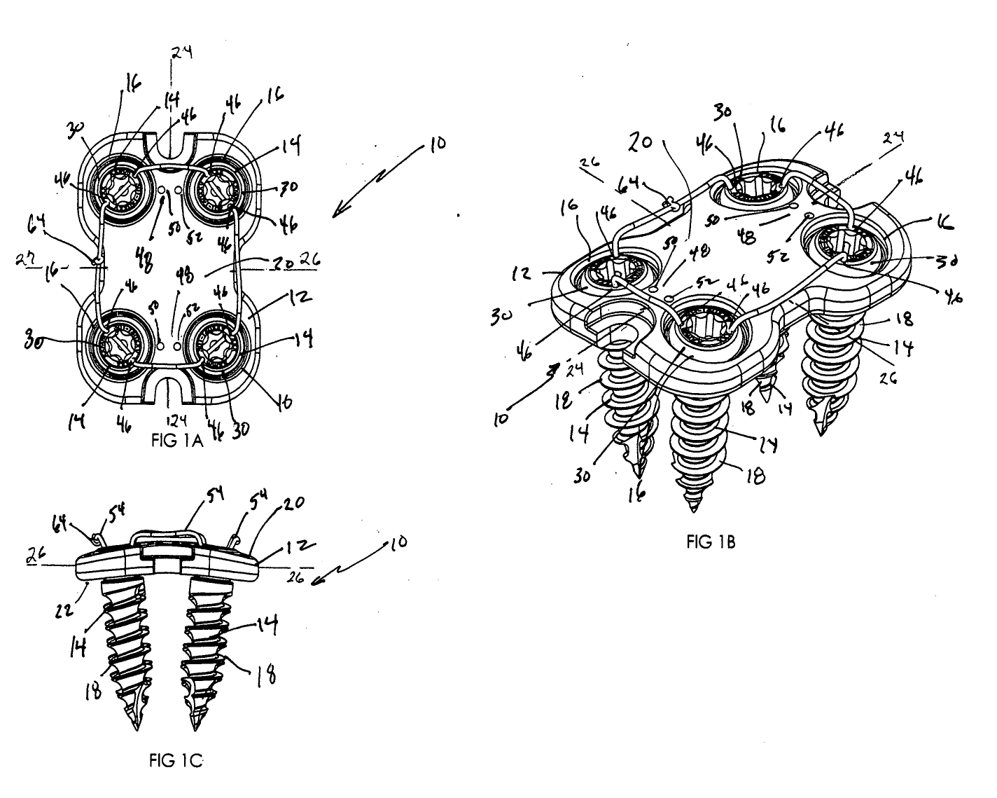

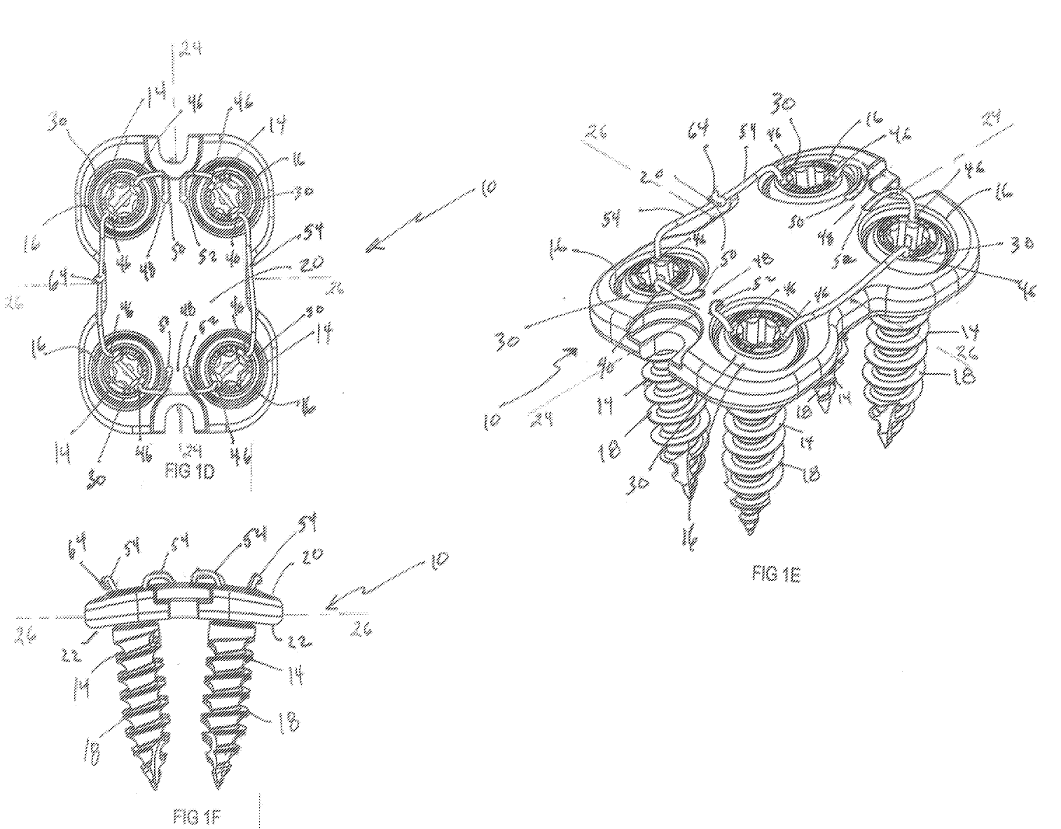

The system, as generally shown at 10 in FIGS. 1A-F, includes a low profile anterior vertebral body plate 12 that, when implanted in a patient, can be secured to the underlying bone using suture lock bone screws 14 as shown in FIGS. 1A-F, 3A-C, and 5A-D. The vertebral body plate 12, as shown in FIGS. 1A-F, and 2A-C, and 6A-D can be provided as an elongated, low profile, plate structure that defines at least one and preferably multiple screw holes 16, which are sized and configured to permit through passage for the threaded portion 18 of the bone screw 14 from the plate upper surface 20 to the plate lower surface 22.

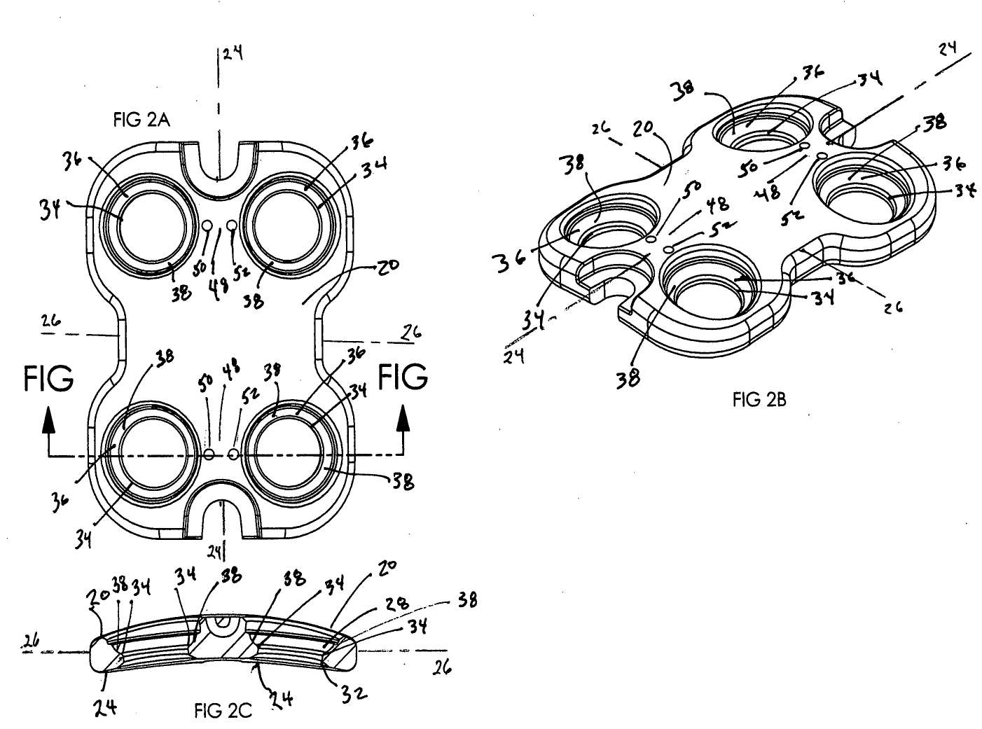

As shown in FIGS. 1A-F, and 2A-C, and 6A-D the plate 12 can be configured to be generally planar; however, the plate preferably will be formed to have arcuate upper and lower surfaces 20, 22, arcing along both the longitudinal axis 24 as well as the transverse axis 26 of the plate 12. This arcing of the plate surface provides a better conformational fit to the anterior surface of the vertebrae to which the plate is to be attached. Each of the screw holes 16, which are defined as through passages in the plate 12, is configured at the upper portion 28 to be generally circular and sized to circumferentially surround the screw head 30 when the screw 14 is fully seated in the plate 12. As shown in FIG. 2C, the lower portion 32 of the screw hole 16 is configured to have an inwardly projecting edge 34, which is formed on its upper side 36 by the generally inward slanting or rounded concave shaped upper side surface 38 of the lower portion 32 of the plate wall defining the screw hole 16. This inward slanting or rounded concave shaped surface 38 can have a complementary shape to that of the underside 40 of the screw head 30. The edge underside 42 is defined by an inward slanting or rounded concave shaped underside surface 44, which is configured to permit limited movement of the threaded portion 18 of the screw 14 during the insertion process so as to provide as necessary a polyaxial relationship of the screw 14 to the plate 12.

As shown in FIGS. 1A,B,D,E, 3A-B and 5A-B, the suture lock bone screw 14 includes at least one suture connecting element 46. The screw suture connecting element 46 is preferably defined within the screw head 30 as a through bore although any connection element capable of providing a connection point on the screw head can be used, such as, for example, hooks, raised or recessed eyelets, posts, or the like.

As shown in FIGS. 1A,B,D,E and 2A-C, a suture plate anchor 48 can be defined on the upper surface 20 of the plate 12. The suture plate anchor 48 is preferably configured as a suture lock through passage having a first opening 50 and a second opening 52 on the plate upper surface 20. It is however within the inventor's concept to configure the suture plate anchor 48 in any form that will facilitate holding or restraining a suture lock at a certain position on the surface of the plate 12, such as, for example, hooks, raised or recessed eyelets, posts, or the like.

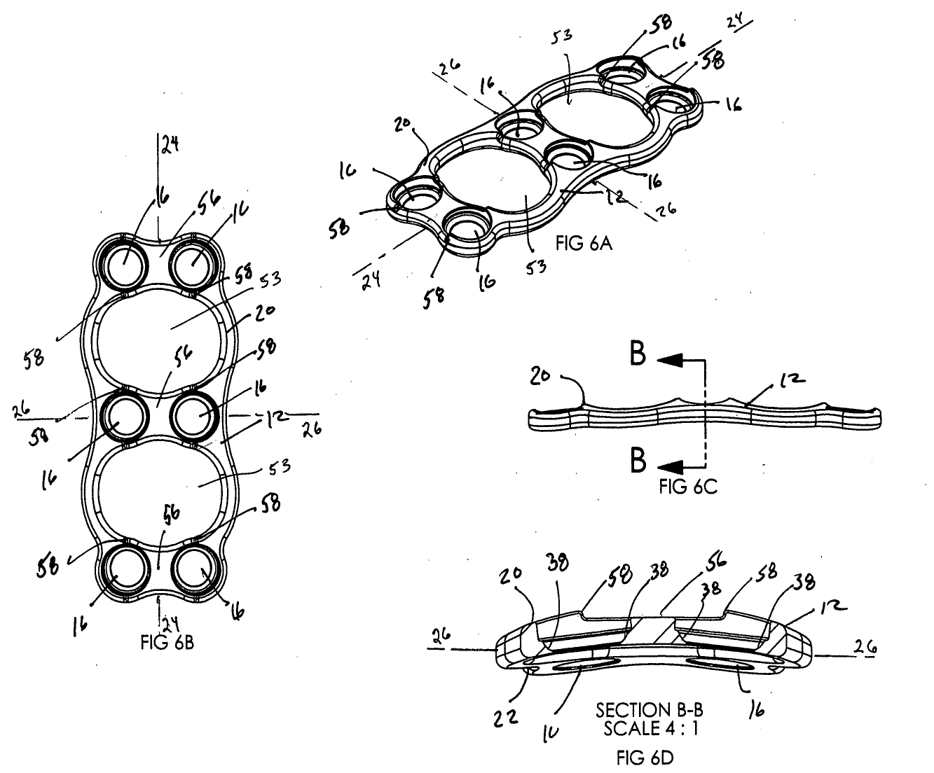

As shown in FIGS. 6A-D, an alternative embodiment of the plate 12, can be provided with a recessed central portion 56 defined in upper surface 20 of the plate 12. The surface of this recessed central portion 56 is below the level of the more lateral portions of the upper surface 20. This alternative plate configuration effectively provides at least one suture lock restricting ledge 58 adjacent to each of the screw holes 16. In practice, the use of this alternative configuration of the plate 12 would allow the surgeon to connect each of the suture lock screws 14 one to the other using a suture lock, which would remain within the lateral limits of the recessed central portion 56 as defined by the restricting ledge 58 thus keeping the suture lock on a plane equal to or below the plane of the upper surface of the plate 12. Such an alternative embodiment of the plate 12 provides the lowest possible profile for the system 10. In addition, the alternative plate configuration shown in FIG. 6A-6D is provided with oversized cut-outs 53, which in comparison to other plates gives the surgeon a larger window through the plate for viewing the disc space beneath. Further, this configuration provides more space between the screw holes 16 thus distributing the force on the plate much like an archway.

As shown in FIGS. 1A-F and 4A-B, the system 10 provides a suture lock 54, which is sized and configured to be easily passed through the screw suture connecting element 46 and the suture plate anchor 48. The suture lock 54 is an elongated flexible surgical suture or wire having a first end and a second end 60, 62, the first end 60 being of such size and configuration so as to facilitate being manipulated or threaded into a locking position through the screw suture connecting element 46 and the suture plate anchor 48. In addition, the suture lock 54 can be fixedly anchored to the plate 48 at any point along the length of the suture lock 54. The suture lock 54 can be manufactured of any flexible, strong, durable, biocompatible material, such as, for example, stainless steel, nickel, titanium, nitinol, tungsten, cadmium, copper, aluminum and alloys of any such materials, nylon, polyester, polyethylene, ultra high molecular weight polyethylene (Spectra), polytetrafluoroethylene, expanded polytetrafluoroethylene, or similar materials. While it is possible to utilize absorbable polymeric materials, including but not limited to glycolide, lactide, caprolactone, dioxanone or like materials as homopolymers or copolymers with each other or with additional materials, it is expected that a non-absorbable material will be preferred for the application of preventing long term backout of screws from bone.

In practice, the plate 12 can be attached to bone by manual insertion of the suture lock screws 14 through the screw holes 16 into the underlying bone material. The inwardly projecting edge 34 permits passage of the threaded portion 18 of the screw 16 through the plate 12; however, it serves to narrow the diameter of the screw hole 16 so as to prevent passage of the screw head beyond the point of contact with the inwardly projecting edge 34. The upper and lower surfaces 36, 42 of the inwardly projecting edge 34 of the screw hole 16 are so configured as to allow the threaded portion 18 of the screw 14 to pass through the plate 12 and at any of a plurality of angles as necessary to best connect to the anatomical conformation of the underlying bone thus providing a polyaxial screw/plate connection.

After the suture lock screws 16 are firmly in place and the plate 12 has thus been secured to the underlying bone, the suture lock 54, as shown in FIGS. 1A-C can be manually, serially threaded through each of the screw suture connecting element 46 of the respective suture lock screws 16. By serially connecting each of the firmly secured screws 16 one to the other, the interactive connection can lock the respective screw heads 30 into a fixed relative position one to the other and thereby effectively prevent the screws 16 from unintentionally backing out of the underlying bone and plate 12. As shown in FIGS. 1D-F, the surgeon can, as deemed necessary, also thread the suture lock 54 in turn through the suture plate anchors 48. This elective additional connection of the suture lock 54 to the suture plate anchors 48 as well as the screw suture connecting element 46 provides an added security to the locking mechanism of the system 10. It is also contemplated that a single screw, or each screw may individually be locked to the plate with individual suture locks threaded through the screw(s) and one or more plate anchors. Upon completion of the threading of the suture lock as desired, the first end and second end 60, 62 of the suture lock 54 can be securely fastened one to the other so as to ensure that the suture lock will remain in place in the system. An example of this fastening of the two ends 60, 62 of the suture lock 54 is show in FIGS. 1A-1F at a suture fastening position 64 located approximately midway along the side of the plate 12. In practice, the fastening position 60 can be located anywhere along the serial circuit to the suture lock 54 as it is threaded through the system 10. The suture ends may be secured to each other by tying, welding, gluing, melt fusing or mechanical coupling (e.g., such as crimping a joining sleeve over the suture ends). It is also contemplated that the suture ends could be secured to the plate, such as by mechanical friction (e.g., a jam cleat jaw), welding, gluing or the like. While it is the preferred embodiment of the system that one suture lock 54 with one fastening position 60 be used, it is also within the conception of the inventors, that multiple suture locks 54 having a corresponding number of fastening positions can be employed; for example, the system can be employed to have one suture lock 54 for each pair of suture lock screws 16 or one suture lock 54 for each set of the screws 16 that are aligned along the two sides of the plate thus providing a system having two relatively parallel threaded suture locks 54. Any such variation of the system 10 disclosed herein is within the conception of the inventors so long as the suture lock 54 effectively secures and limits the unintentional backing out of the screws 16 from the plate 12 and the underlying bone. Advantageously, the system 10 requires no additional small locking elements as are used in many conventional efforts to provide a locking method for vertebral plate devices.

When determined to be necessary or desirable, the plate 12 can be removed from its secured and locked position against the bone by severing and un-threading the suture lock 54 and subsequently manually applying reverse torque to the suture lock screws 16.

The above described method of use of the system 10 can be employed as a method of stabilizing or fixing injured or diseased vertebrae and if necessary, multiple devices or a device, which is elongated beyond the examples depicted herein, can be employed as necessary.

While the device as described herein can be preferably used to attach to the anterior surface of cervical vertebrae and is configured to be capable of stabilizing cervical vertebrae, it is within the inventors' understanding that the plate can be configured and adapted to conform to any implantable surgical plate requirement to provide a low profile plate capable of securing and stabilizing any injured or diseased bone.

The device 10 can be manufactured as integral components by methods known in the art, to include, for example, molding, casting, forming or extruding, and machining processes. The components can be manufactured using materials having sufficient strength, resiliency and biocompatibility as is well known in the art for such devices. By way of example only, suitable materials can include implant grade metallic materials, such as titanium, cobalt chromium alloys, stainless steel, or other suitable materials for this purpose. It is also conceivable that some components of the device can be made from plastics, composite materials, and the like.

It is also within the concept of the inventors to provide a kit, which includes at least one of the vertebral plate and suture lock screw systems disclosed herein. The kit can also include additional orthopedic devices and instruments; such as for example, instruments for tightening or loosening the bone screws, spinal rods, hooks or links and any additional instruments or tools associated therewith. Such a kit can be provided with sterile packaging to facilitate opening and immediate use in an operating room.

Each of the embodiments described above are provided for illustrative purposes only and it is within the concept of the present invention to include modifications and varying configurations without departing from the scope of the invention that is limited only by the claims included herewith.

What is claimed is:

1. A novel bone plate system, comprising:
A plate having an upper surface and a lower surface, said plate defining at least two bone screw holes, said bone screw holes having a generally circular configuration,
at least two suture lock bone screws corresponding to said at least two bone screw holes, said suture lock bone screws having a screw head and a threaded portion, said screw head defining on its upper surface at least one screw suture connecting element,
at least one flexible, elongated suture lock sized and configured to be capable of connecting to said screw suture connecting element on each of said suture lock bone screws,
wherein said at least two suture lock bone screws connected to one another by said elongated suture lock are restricted from unintentionally backing out of said plate.

2. The bone plate system of claim 1, wherein said suture lock bone screw is capable of polyaxial alignment with said plate.

3. The bone plate system of claim 1, wherein said plate is an anterior vertebral body plate.

4. The bone plate system of claim 1, wherein said bone screw suture connecting element is formed by machining.

5. The bone plate system of claim 4, wherein said bone screw connecting element is a through bore sized and configured to allow slidable through passage of said elongated suture lock.

6. The bone plate system of claim 1, wherein said suture lock bone screw comprises two screw suture connecting elements.

7. The bone plate system of claim 1, wherein said plate is configured to have an upper and lower curved surface, said curve being along the longitudinal axis of the plate.

8. The bone plate system of claim 1, wherein said plate is configured to have an upper and lower curved surface, said curve being along the transverse axis of the plate.

9. The bone plate system of claim 7, wherein said curve is also along the transverse axis of the plate.

10. The bone plate system of claim 1, wherein each of said screw holes comprise an inwardly projecting edge between the upper and lower portion of the bone screw hole, said inwardly projecting edge serving to decrease the diameter of said holes at the position of said edge to a size larger than the diameter of said threaded portion of said bone screws and smaller than the diameter of the head of said bone screws, such that said threaded portion of said screw can freely pass through said bone screw hole and said head of said bone screw is restricted from passing all the way through the bone screw hole.

11. The bone plate system of claim 10, wherein said edge has an upper surface with a concave conformation and said bone screw head has an underside with a convex conformation complimentary to concave conformation of said edge upper surface, such that said underside of said bone screw head and said upper surface of said edge are capable of articulating one with the other in a polyaxial manner during the process of screw insertion into said plate.

12. The bone plate system of claim 1, wherein said plate further comprises at least one suture lock anchor sized and configured to receive said elongated suture lock.

13. The bone plate system of claim 12, wherein said suture lock anchor is defined in the upper surface of said plate.

14. The bone plate system of claim 13, wherein said suture lock anchor is a suture lock through passage having a first opening and a second opening defined on the plate upper surface.

15. A bone plate system, comprising:
a plate having an upper surface and a lower surface, said plate defining at least one bone screw hole and at least one suture plate anchor,
at least one suture lock bone screw said suture lock bone screw having a screw head and a threaded portion, the threaded portion configured and dimensioned to be inserted through the at least one bone screw hole and the screw head configured and dimensioned to seat within said bone screw hole, said screw head defining on a portion thereof at least one screw suture connecting element,
at least one flexible, elongated suture lock sized and configured to be capable of connecting to said screw suture connecting element on said suture lock bone screw and to said suture plate anchor, whereby said suture lock bone screw is restricted against unintentionally backing out of said plate.

16. A method of stabilizing a vertebral body, the method comprising,
providing a bone plate having a plurality apertures, each said aperture configured and dimensioned to receive a bone screw,
surgically accessing a surface of a vertebral body,
positioning said bone plate and attaching same to said vertebral body using a plurality of bone screws inserted through the apertures to engage bone, at least two of said bone screws each having a suture lock connecting member defined thereon,
providing at least one elongated suture lock,
connecting at least two of said suture lock screws one to the other with said elongated suture lock and securing the ends of said elongated suture lock such that said connected suture lock screws cannot unintentionally back out of said plate.

17. The method of claim 16 further comprising the step of securing the ends of the elongated suture lock to each other.

18. The method of claim 16 further comprising securing the ends of the elongated suture lock to the plate.

19. A method of stabilizing a vertebral body, the method comprising,
providing a bone plate having at least one apertures configured and dimensioned to receive a bone screw and at least one bone plate suture anchor,
surgically accessing a surface of a vertebral body,
positioning said bone plate and attaching same to said vertebral body using at least one bone screws inserted through the at least one aperture to engage bone, said bone screw having at least one suture lock connecting member defined thereon,
providing at least one elongated suture lock,
connecting said elongated suture lock to said suture lock connecting member and to said bone plate suture anchor such that said bone screw cannot unintentionally back out of said plate.

20. A kit comprising:
at least one bone plate having at least one bone plate suture anchor and at least one aperture configured and dimensioned to receive a bone screw,
at least one bone screw having a threaded portion and a head portion, the threaded portion configured and dimensioned to be inserted through the bone plate aperture and the screw head configured and dimensioned to be seated in the bone plate aperture, each said bone screw having at least one suture lock connecting member defined thereon.

21. The kit according to claim 20 further comprising at least one elongated suture lock.

22. The kit according to claim 20 further comprising a plurality of said bone plates and said bone screws.

23. The kit according to claim 22 further comprising at least one elongated suture lock.

24. The kit according to claim 20 further comprising at least one instrument for implanting said bone plate and said bone screw.
